(19) United States
(12) Patent Application Publication
(10) Pub. No.: US 2023/0204564 A1
(43) Pub. Date: Jun. 29, 2023

(54) SYSTEM FOR DETECTING BIOLOGICAL VIRAL PARTICLES EMITTED INTO THE AIR BY A LIVING BEING

(71) Applicant: Marco DE ANGELIS, CESENA (IT)

(72) Inventor: Marco DE ANGELIS, CESENA (IT)

(21) Appl. No.: 17/920,176

(22) PCT Filed: Mar. 9, 2021
(86) PCT No.: PCT/IT2021/050054

§ 371 Date: Oct. 20, 2022

(30) Foreign Application Priority Data
Apr. 23, 2020 (IT) 102020000008701

(51) Int. Cl. G01N 33/497; A61B 5/097; G01N 1/40

(52) U.S. Cl. CPC G01N 33/497; A61B 5/097; G01N 1/4022; G01N 2001/4033

(57) ABSTRACT

The present invention relates to a system for detecting biological viral particles emitted into the air by a living being by means of an expiratory air volume, comprising at least one detecting and processing device of said expiratory air volume, capable of detecting a sample of said expiratory air volume and of condensing it into a condensed sample to be analyzed, and at least one analyzing device, connected to said detecting and processing device, capable of receiving said condensed sample, of detecting electrical signals associated with said condensed sample and of processing said electrical signals, so as to detect the viral biological particles contained therein.

### FIELD

The present invention relates to a system for detecting biological viral particles emitted into the air by a living being, in particular in an air sample.

More in detail, the invention relates to a system of the above type, studied and implemented in particular for the detection of biological particles of a viral nature emitted into the air by a living being, but which can be used for any type of biological particles, of which it is necessary the detection of their presence in a gaseous fluid sample.

In the following, the description will be directed to the detection of biological particles of a viral nature, present in a sample of air exhaled by a living being, but it is clear that the same should not be considered limited to this specific use.

### BACKGROUND

As is well known, currently the search for biological particles, in particular when considered dangerous, such as, for example, those of pathogenic microbes, such as viruses, is normally carried out in a subject considered to be a potential host of the biological entity sought, through various methods, such as culture, serological tests or the well-known PCR
Polymerase Chain Reaction technique on a sample taken from the mucous membranes, through a swab, or from the blood, by sampling.

Alternatively, to evaluate a current or previous contact of a subject with a pathogen, specific antibodies in the blood are also searched, directly or indirectly.

These known methods present various problems, often coexisting, such as, for example: the need for the intervention of specialized personnel, both in the sampling as well as in the analysis; the discomfort suffered by the subject for some sampling methods; the transfer of the sample from the sampling site to the analysis site; the possible filtering and/or treatment of the sample; the time required to perform the analysis; the use of reagents; the total cost due to one or more of the previously mentioned known methods.

These criticalities can reduce the use of these methods, slow down the response time, and, consequently, that of intervention, such as isolating the positive subject, thus facilitating the spread of the infecting agent and the contagion.

In the infection process of an organism by a pathogen, a particularly important variable is the infectious charge, i.e., the number of infectious particles with which the organism comes into contact.

For many diseases, such as, for example, influenza viruses or the like, the contact of a high number of infectious particles with the mucous membranes of the airways can, other things being equal, more likely result in a disease, overwhelming the antibody response of the organism.

On the contrary, contact with a low number of infectious particles can more likely result in a paucisymptomatic or even asymptomatic contagion, as the antibody response is able to equalize the causes of the contact, in the first case, or overcome them, in the second case, but however, being able to make the subject immune to the infectious agent.

The viral load released by the contagious subject and introduced by the still healthy subject is therefore positively correlated, other conditions being equal, such as the state of health of the healthy subject, age, coexisting or previous pathologies, the probability of getting sick and the severity of the disease.

Considering the infectious process as a whole, i.e., as a struggle between two organisms, the infecting one and the one that must defend itself from the infection, the target of the infecting organism, in the case of respiratory viruses, which need physical conditions to be able to replicate, chemical and biological of a higher organism, is to invade the host organism, overcoming its defenses, in order to replicate itself to the maximum extent and, once the maximum number of replicas has been reached, move to another subject to continue replication.

In this path, the mucous membranes are the potential gateway for the virus to enter the body but also the structures in which, being richer in defenses, the body seeks, thanks to the turbulent flows that the structures of the upper respiratory tract create to the incoming air, to block the viruses before they reach the deepest and most vulnerable structures of the body and, possibly, destroy them, by means of the antibodies and substances present there.

Therefore, the presence of viral material in the mucous membranes is not necessarily a signal of the contagiousness of a subject, as they may be particles deposited there during inhalations, in particular inhalations of air rich in viruses, or viruses already destroyed by the local immune defenses, therefore no longer active but still detectable as genetic or antigenic material, which are indistinguishable from the active virus.

This therefore implies, for example, that the material detected by the swabs used by the known methods may concern viruses that are already inactivated, and which therefore can provide false-positive results.

The positive subject to the mucosal swab could therefore actually be in different stages: both at the first contact with the virus, both asymptomatic sick, and not yet symptomatic sick, both symptomatic, both cured and immunized but on whose mucous membranes have just deposited viruses not yet neutralized or already neutralized.

On the other hand, when a virus has really infected an organism, that is, when it has reached the deepest parts of its respiratory tree, replicating itself in the journey from cell to cell, it must necessarily leave the infected body to go and look for other bodies, where it continues to replicate.

Since the pulmonary alveoli lack a defensive mucous layer to pass through, this time, in the opposite direction, the virus then passes directly from the cells to the alveolar air and, favored by the fact that the host organism benefits from an outgoing airflow as laminar as possible, in order not to have unnecessary resistance to the outgoing flow, which must not impact on the mucous membranes, leaves the body, in large numbers, in the exhaled air.

In any case, if already in the trachea and bronchi, the virus, having invaded the mucous cells of the corresponding tracts and having destroyed them, depriving them of the mucous barrier, can pass outside the respiratory tract and thus be conveyed, during exhalation, towards the outside the body.

The exhaled air, therefore, contains the viruses exiting the body when the virus has already been able to replicate enough to damage the mucous cells of the more or less deep sections of the respiratory tract, right into the lungs and, therefore, when the subject is really contagious.

The viral load in the exhaled air, that is the density of the viral particles, also indicates the level of contagiousness of the subject, and can therefore allow to classify the subjects according to this level of contagiousness and, finally, better isolate only those who are really contagious.

Exhaled air is therefore a better indicator than mucus and blood of a person's contagious state and level of contagiousness.

A second problem is further known.

Many pathogenic viruses for humans can transit, from asymptomatically to symptomatically, in other species of animals including domestic ones.

The examination of mucous or blood swabs in all domestic animals is an extremely expensive and impractical procedure, thus losing the possibility of identifying the virus and following its epidemic spread.

Another problem concerns the fact that a subject can easily become infected by breathing in closed air densely packed with viral particles emitted by other contagious subjects.

The determination of the density of viral particles per volume of ambient air is a reliable indicator of its healthiness, i.e., in the absence of viral particles, or not, i.e., when there is the presence of viral particles and, in the case of presence, an indicator of its danger in generating other sick people.

This determination can therefore be, for example, a system for regulating the ventilation system in a health facility, where contagious subjects transit or are hosted.

### SUMMARY

In the light of the above, it is, therefore, an object of the present invention to provide a system for detecting viral biological particles, and for measuring their density in a sample of volume of air exhaled by a living being, during exhalation.

Another object of the invention is to provide a reliable and economical system for the detection of viral biological particles and for the measurement of their density in the ambient air.

It is therefore specific object of the present invention a system for detecting biological viral particles emitted into the air by a living being by means of an expiratory air volume, comprising at least one detecting and processing device of said expiratory air volume, capable of detecting a sample of said expiratory air volume and of condensing it into a condensed sample to be analyzed, and at least one analyzing device, connected to said detecting and processing device, capable of receiving said condensed sample, of detecting electrical signals associated with said condensed sample and of processing said electrical signals, so as to detect the viral biological particles contained therein.

Further according to the invention, said detecting and processing device

comprises a conveying element of said expiratory air volume, which can be put on the head of said living being, capable of receiving at the entry said expiratory air volume and of sending it at the exit, and a condensing element, connected to said conveying element, capable of receiving said expiratory air volume and to cause its condensation into a condensed sample.

Preferably according to the invention, said condensing element is of the single-use type and/or unidirectional type.

Still according to the invention, said detecting and processing device comprises a measuring member of said expiratory air volume capable of timing the detection of said expiratory air volume or capable of measuring the breaths of said living being.

Always according to the invention, said measuring member is arranged internally or externally to said conveying element.

Further according to the invention, said detecting and processing device comprises an acoustic and/or visual signaling element, capable of signaling the reaching of the minimum expiratory air volume to be sampled, or the minimum time or the minimum number of breaths to reach said volume.

Preferably according to the invention, said detecting and processing device comprises a processing cell of said condensed sample.

Still according to the invention, said conveying element is an airtight mask, capable of being placed on the face or head of a human or animal living being.

Always according to the invention, said conveying element is a mouthpiece which can be put in the mouth of a living being.

Further according to the invention, said conveying element is a suction provided with an entry opening, into which said expiratory air volume enters, an exit opening, from which said expiratory air volume comes out, and comprising internally an air moving device, capable of facilitating the entry of said expiratory air volume through said entry opening.

Preferably according to the invention, said analyzing device comprises a detecting module, comprising in its turn a support on which the condensed sample can be placed, provided with a plurality of sensors of the type biosensors or nano-sensors or nano-pores, and a processing module, connected to said detecting module, comprising a plurality of processing channels and a processor, in which said plurality of processing channels is connected to said plurality of sensors, capable of detecting electrical signals and of sending said electrical signals to said processor, for detecting said biological viral particles.

### BRIEF DESCRIPTION OF THE FIGURES

The present invention will be now described, for illustrative but not limitative purposes, according to its preferred embodiments, with particular reference to the figures of the enclosed drawings, wherein:

FIG. 1 illustrates a schematic view of a system for detecting viral biological particles emitted into the air by a living being, object of the present invention;

FIG. 2 illustrates a side schematic view of a component of the system shown in FIG. 1;

FIG. 3 illustrates a side schematic view of a second embodiment of the component shown in FIG. 2; and

FIG. 4 illustrates a schematic front view of a third embodiment of the component shown in FIG. 2.

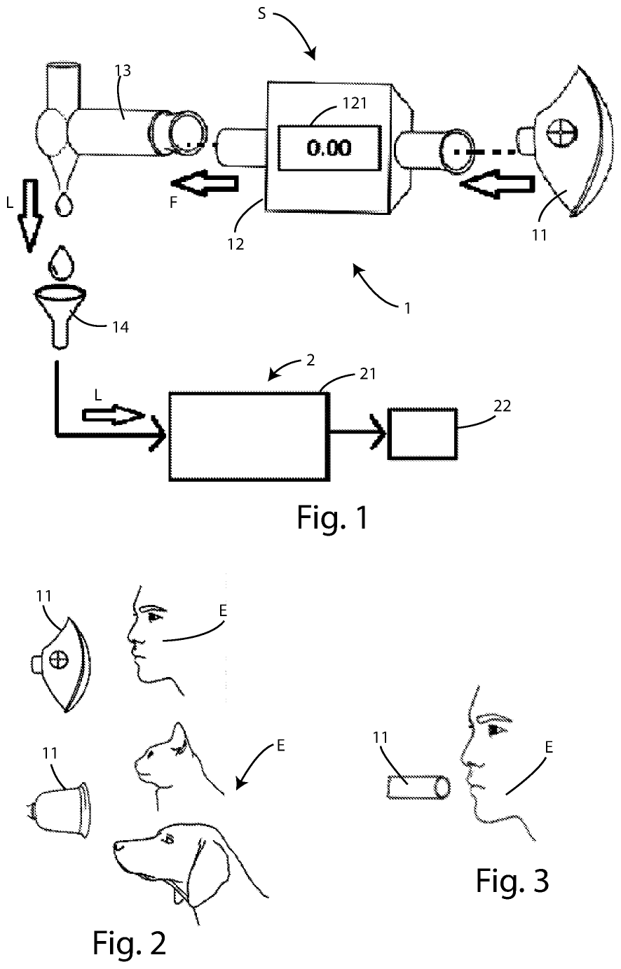

### DETAILED DESCRIPTION OF THE EMBODIMENTS

In the various figures similar parts will be indicated with the same numerical references.

With reference to the attached figures, the system S for detecting viral biological particles emitted into the air by a living being E, object of the present invention, essentially comprises a device for detecting and treating the sample of air or air volume F to be analyzed, emitted during the exhalation of a living being E, and an analysis device 2 of the detected air sample.

Said detection and treatment device 1 is capable of sampling a quantity of the air volume F exhaled by a living being E.

Said device 1 comprises an element 11 for conveying the air volume F, a member 12 for measuring said air volume F, a condensing element 13 for the liquid content dispersed in said volume of sampled air F, and a processing cell 14 of the condensed.

In particular, with reference to FIG. 2, said conveying element 11 is a hermetically sealed mask, capable of being placed on the face or head of a living being E, so as to hermetically isolate it from the external environment.

Said mask 11 can be used both for a living being E of human and animal type, suitably adapting the external structure to the physical conformation of the living being E.

Said conveying element 11 comprises within a one-way valve, not shown in the figure, for conveying said air volume F from the mouth of the living entity E towards the subsequent elements of the detection and treatment device 1.

Said conveying element 11 can preferably be of the disposable type, to avoid the contagion risk of living beings E tested in succession with each other.

In the case of living beings E already ill, subjected to ventilation by means of closed circuits, the withdrawal of said volume of exhaled air F is carried out directly in the circuit where the exhaled air of these living beings E is collected.

Therefore, the measurement of the quantity of viral particles emitted with the exhaled breath, at a certain moment, or in the exhaled air collected, in case of the living beings E are connected to closed circuits, and the variations, presumably in crescendo-decrescendo of this quantity, can also constitute a criterion for following the evolution of the disease.

Said member 12 for measuring the air volume F receives at its inlet said air volume F exiting from said conveying element 11.

Without departing from the scope of protection of the present invention, said measuring member 12 can also be inserted inside said conveying element 11.

Said measuring member 12 can be a flow meter, or a timer, or a number of breaths meter.

Said measuring member 12 is provided with an acoustic and/or visual signaling element 121, so as to signal to the operator responsible for the use of said system S, that the minimum quantity of air volume F to be sampled has been reached, necessary or, alternatively, the minimum time or, alternatively, the minimum number of breaths to reach this air volume F.

Said measuring member 12 is preferably disposable to ensure medical safety.

Or, alternatively, said measuring member 12 is unidirectional, so as to ensure the impossibility of inversion of the air volume F, from the outside towards the living entity E.

Said condensing element 13 is connected to said measuring member 12.

In the embodiment wherein said measuring member 12 is comprised within said air conveying element 11, said condensing element 13 is connected directly to said conveying element 11.

Said condensing element 13 is capable of receiving the volume of exhaled air F and to condense in the liquid phase the water vapor naturally contained in said volume of exhaled air F, which therefore becomes a condensed sample L.

Said condensed sample L is then transmitted to the subsequent analysis stages.

Said condensing element 13 carries out the condensation of the water vapor by means of known methods.

In particular, the condensation takes place either through a direct cooling of the exhaled volume of air F, or through the contact of the exhaled volume of air F with a material, which allows its condensation, such as, for example, a metal sheet, possibly cooled.

Said condensing element 13 is of the disposable type or, alternatively, it must guarantee the impossibility of inversion of the air volume F to further reduce the possibility of contagion of the next tested living being E.

Said condensate processing cell 14 is connected to said condensation element 13, and it is capable of receiving the condensed sample L at the inlet and carrying out treatments on it.

In particular, a chemical and/or physical and/or microbiological treatment can be carried out on said condensate L in order to make it easier to analyze in the subsequent phases.

By way of example, the treatment could consist of filtering and/or treating with antibodies to identify and then make the microbial particles more visible.

However, the presence of said processing cell 14 in said detection and treatment device 1 is optional.

With reference to FIG. 3, in a second embodiment, said conveying element is a mouthpiece 11′ to be inserted in contact with the, or in the mouth of a human being E.

The above description for said conveying element 11 is also applied unchanged to said mouthpiece 11′.

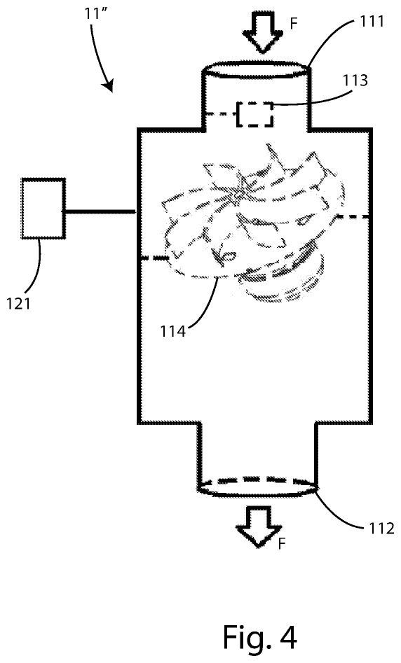

With reference to FIG. 4, in a third embodiment, said conveying element is a suction 11″.

Said suction 11″ is a hollow container, preferably having a cylindrical shape, but which can nevertheless have different shapes and sizes.

Said suction 11″ is provided with an inlet opening 111 and an outlet opening 112.

Said air volume F flows into said inlet opening 111, while said air volume F comes out from said outlet opening.

Each of said inlet opening 111 and outlet opening 112 can be hermetically sealed.

Furthermore, each of said inlet opening 111 and outlet opening 112 can be closable manually or automatically.

A humidifier 113 is arranged within said suction device 11″, which is capable of humidifying said volume of incoming air F with a known concentration of water vapor per volume of air, so as to achieve the formation of a minimum condensed quantity for the analysis of the particle density per volume of ambient air.

Inside said suction 11″ there is also an air handling device 114, in particular a fan 114, capable of facilitating the entry of the air volume F through said inlet opening 111.

Said air movement device 114, as well as the opening of said suction 11″, can be operated manually or by means of a timer, or on the basis of certain factors such as: the number of living beings E present in a given environment, the achievement of certain concentrations of gas, which is an indirect measure of the air exhaled by living beings E present in the environment, and/or the type of physical activities carried out in the environment.

All these factors are potentially connected with a greater or lesser presence of potentially harmful biological particles emitted with the breath of living beings and sick and therefore contagious.

The description of said conveying element 11 remains unchanged also for the third embodiment of said suction 11″, and differs only in the following characteristics.

Said measuring member 12, if internally arranged, can be arranged in proximity of said inlet opening 111 or in proximity of said outlet opening 112, so as to measure the volume of air transited per unit of time.

Furthermore, said conveying element 11″ can be directly connected to said signaling element 121, as shown in FIG. 4.

Said analysis device 2 is connected to said detection and treatment device 1 by means of the processing cell 14, otherwise, in the absence of said processing cell 14, it is connected directly to said condensation element 13.

In the case of the third embodiment, said analysis device 2 can be connected directly to said outlet opening 112.

Said analysis device 2 can be of various substantially known types, the preferred ones are described.

Said analysis device 2 is preferably of the type comprising sensors, biosensors, or nano-sensors, by means of which it is possible to carry out detection and a subsequent analysis of ion channels, which are responsible for the exchange of trans-membrane proteins between intra and extra-cellular environments.

The ion channels are therefore able to respond to chemical-physical stimulations.

Said analysis device 2 essentially comprises a detecting module 21 and a processing module 22, connected to said detecting module 21.

In particular, said detecting module 21 comprises a support or plate of inorganic type material, preferably SiO₂ or SiNₓ, on which nano-sensors or biosensors or artificial nano-pores of nano-metric dimensions, from a few nm up to hundreds of nm, for example 10⁻⁹ m, which come into contact with said condensed sample L to be examined.

Said processing module 22 comprises a plurality of processing channels connected to said detecting module 21, in particular, to said nano-pores support and to a processor.

When said condensed sample L comes into contact with said support, due to microfluidicity phenomena, said condensed sample L comes into contact with said nano-pores.

Consequently, suitable electrical signals are detectable by said plurality of processing channels, which send said electrical signals to said processor for processing said electrical signals.

In particular, said nano-pores detect the nano-metric particles with which they interact.

An interaction mode can, for example, be the following.

Said support, having nano-pores of the desired size, can be arranged as a separating element between two compartments containing an electrolytic solution, capable of sustaining an electric current, the flux variations of which can be measured.

If material containing nano-particles to be detected, such as viral particles or viral particles sensitized with modifications of their characteristics, is introduced into one of the two compartments, their passage through the nano-pores of the specific size can alter the flow of current.

These alterations, which can be associated with the characteristics of the nano-particles, such as, for example, mass, shape, and size, can be measured by said processing module 22 in real-time.

Said processing module 22 provides, in a digital and/or analog form and/or when thresholds are crossed, the results of the qualitative and quantitative analysis of the fluid sample L, showing whether the researched particles are present, presumably beyond a certain minimum threshold, and, in the positive case, with which quantitative presence, for example per unit of volume of the sample and, knowing the ratio of the fluid sample to the volume of air, per unit of volume of air subject to sampling.

The particles can be detected thanks to the nano-pores by various known techniques.

The two preferred methods are substantially described.

The first way is of the protein-protein interaction type in the nano-pore.

The detection principle is based on the interaction between the antibodies attached to the surfaces of the nano-pore, or functionalization, and the proteins present on the particle to be detected, such as, for example, in the case of the Corona Virus 19, the S1-spike on its capsid.

As the potential of virus particles present in said condensed sample L is intercepted by the antibodies in the nano-pore, the measured current is reduced depending on the number of particles, which remain attached to the nano-pore, contributing to its partial occlusion.

This technique has the advantage of being able to be used directly on a condensed sample L, without the pretreatment to sensitize them operated by said processing cell 14.

Furthermore, the presence of specific antibodies able to bind only to the searched particles ensures their selectivity with respect to other possible particles with very similar characteristics present in the sample.

The second mode is of the Resistive Pulse Sensing type.

This technique is based on the analysis of the shape of the current signal generated during the passage of the specific particle sought through the nano-pore.

For example, if the average diameter of a virus, excluding spikes, is between 82 and 94 nm, as in the case of the Corona Virus, using a nano-pore with a size of 300 nm and analyzing the impulses generated by the transit of the virus within it, it is possible to correlate the characteristics of the current pulses with the specific virus.

The main limitation of this technique is the lower specificity, due to the less exact discrimination of the type of particles, for example the viral ones of a certain virus, if other particles, viral or not, of similar size are present in the sample.

However, this technique can be used, taking advantage of fewer steps and less complexity and therefore shorter times and lower costs, in a first step of evaluation of the presence of all particles of a certain type, such as, for example, of all viruses of a certain family, in order to eventually be able to discriminate, in subsequent passages, which type it is.

As is evident from the above description, said detection system S is able to quickly and reliably detect the presence of viral biological particles in a sample of air exhaled by a living being, both by taking the sample directly from the living being, and indirectly from the environment in which several subjects are, or have been present, at a given time.

The present invention has been described for illustrative but not limitative purposes, according to its preferred embodiments, but it is to be understood that modifications and/or changes can be introduced by those skilled in the art without departing from the relevant scope as defined in the enclosed claims.

1-11. (canceled)

12. A system for detecting biological viral particles emitted into the air by a living being by an expiratory air volume, comprising:
at least one detecting and processing device configured for detecting a sample of the expiratory air volume and condensing the sample into a condensed sample to be analyzed; and
at least one analyzing device connected to the detecting and processing device, configured for receiving the condensed sample, detecting electrical signals associated with the condensed sample and processing the electrical signals so as to detect viral biological particles contained within the condensed sample.

13. The system according to claim 12, wherein the detecting and processing device further comprises;
a conveying element configured to be put on a head of the living being, the conveying element comprising an entry configured for receiving the expiratory air volume, and an exit capable of sending the expiratory air volume, and
a condensing element connected to the conveying element, wherein the condensing element is configured for receiving the expiratory air volume and of condensing the expiratory air volume into the condensed sample.

14. The system according to claim 13, wherein the condensing element is single-use and/or unidirectional.

15. The system according to claim 13, wherein the detecting and processing device further comprises a measuring member configured for timing the detection of the expiratory air volume or measuring the breaths of the living being.

16. The system according to claim 15, wherein the measuring member is arranged internally or externally to the conveying element.

17. The system according to claim 12, wherein the detecting and processing device further comprises an acoustic and/or visual signaling element configured for signaling when the minimum expiratory air volume to be sampled is reached, or the minimum time or the minimum number of breaths to reach the minimum expiratory air volume.

18. The system according to claim 13, wherein the detecting and processing device further comprises a processing cell of the condensed sample.

19. The system according to claim 13, wherein the conveying element is an airtight mask configured to be placed on the face or head of the living being.

20. The system according to claim 13, wherein the conveying element is a mouthpiece configured to be put in a mouth of the living being.

21. The system according to claim 13, wherein the conveying element is a suction element comprising;
an entry opening into which the expiratory air volume enters,
an exit opening from which the expiratory air volume comes out, and
an internal air moving device, configured for facilitating the entry of the expiratory air volume through the entry opening.

22. The system according to claim 12, wherein the analyzing device further comprises:
a detecting module, comprising a support on which the condensed sample can be placed and including a plurality of sensors, wherein the sensors are biosensors, nano-sensors, or nano-pores sensors, and
a processing module connected to the detecting module, the processing module comprising a plurality of processing channels and a processor, wherein the plurality of processing channels are connected to the plurality of sensors and are configured for detecting electrical signals and of sending electrical signals to the processor, wherein the electrical signals detect biological viral particles.
